(19) United States
(12) Patent Application Publication
(10) Pub. No.: US 2025/0160988 A1
(43) Pub. Date: May 22, 2025

(54) Irrigation Drape Assembly

(71) Applicant: Benjamin Todd Raines, Stuart, FL (US)

(72) Inventor: Benjamin Todd Raines, Stuart, FL (US)

(21) Appl. No.: 18/955,317

(22) Filed: Nov. 21, 2024

Related U.S. Application Data
(60) Provisional application No. 63/601,376, filed on Nov. 21, 2023.

(51) Int. Cl. A61B 46/20; A61M 1/00

(52) U.S. Cl. CPC A61B 46/20; A61M 1/69; A61M 1/884; A61B 2046/201; A61B 2046/205

(57) ABSTRACT

An irrigation drape assembly includes: a drape portion configured to be positioned beneath a body portion on which a medical procedure is being performed; a collection portion configured to collect irrigation fluid produced during the medical procedure on the body portion, thus defining collected irrigation fluid; a evacuation portion coupled to the collection portion and configured to receive the collected irrigation fluid and direct the collected irrigation fluid toward an evacuation port included in the evacuation portion; and a straining assembly configured to strain solids/semisolids from the collected irrigation fluid and prevent the solids/semisolids from entering the evacuation port, thus defining strained irrigation fluid.

### RELATED APPLICATION(S)

This application claims the benefit of U.S. Provisional Application No. 63/601,376, filed on 21 Nov. 2023, the entire contents of which are herein incorporated by reference.

### TECHNICAL FIELD

This disclosure relates to irrigation drape assemblies and, more particularly, to irrigation drape assemblies that collect irrigation fluid.

### BACKGROUND

The history of surgical irrigation drapes designed to collect irrigation fluid has evolved significantly as operating room demands and technology have advanced. Early surgical drapes, typically cloth, provided limited fluid control, requiring surgical teams to use additional tools and towels for excess fluids. However, as surgery techniques became more complex, the need for specialized draping solutions emerged, particularly for surgeries involving heavy irrigation like orthopedics and arthroscopy.

Modern surgical irrigation drapes incorporate features like fluid-collection pouches, absorptive materials, and even direct-to-drain connections for high-volume fluid management. For example, some drapes are outfitted with adhesive strips to prevent slippage, elastic fenestrations for precise coverage, and multiple exit ports to facilitate continuous drainage. These innovations help improve fluid management, minimize contamination risks, and support a sterile environment by reducing fluid spillage and absorption on the operating room floor.

Manufacturers like 3M and others have developed these drapes with specialized features tailored to specific surgical fields, enhancing both patient safety and operational efficiency. Some options even incorporate antimicrobial films, such as 3M's Ioban, to provide additional barriers against microbial contamination in high-risk procedures.

### SUMMARY OF DISCLOSURE

In one implementation, an irrigation drape assembly includes: a drape portion configured to be positioned beneath a body portion on which a medical procedure is being performed; a collection portion configured to collect irrigation fluid produced during the medical procedure on the body portion, thus defining collected irrigation fluid; a evacuation portion coupled to the collection portion and configured to receive the collected irrigation fluid and direct the collected irrigation fluid toward an evacuation port included in the evacuation portion; and a straining assembly configured to strain solids/semisolids from the collected irrigation fluid and prevent the solids/semisolids from entering the evacuation port, thus defining strained irrigation fluid.

One or more of the following features may be included. The body portion may be a limb on which the medical procedure is being performed. The drape portion may include: a limb dam portion. The limb dam portion may include: a limb passage for at least partially surrounding the limb on which the medical procedure is being performed. The limb dam passage may include: an elastic cinching assembly for cinching around the limb on which the medical procedure is being performed. The collection portion may be coupled to the drape portion along a single edge of the drape portion. The collection portion may be coupled to the drape portion along multiple edges of the drape portion. The straining assembly may include: a fenestrated straining layer configured to strain the solids/semisolids from the collected irrigation fluid and prevent the solids/semisolids from entering the evacuation port, thus defining the strained irrigation fluid. The solids/semisolids in the captured irrigation fluid may include biological solids/semisolids. The evacuation port may be configured to direct the strained irrigation fluid into an external container assembly. The evacuation port may be configured to be releasably coupleable to an external fluid collection device.

In another implementation, an irrigation drape assembly includes: a drape portion configured to be positioned beneath a limb on which a medical procedure is being performed, the drape portion including a limb dam portion; a collection portion configured to collect irrigation fluid produced during the medical procedure on the limb, thus defining collected irrigation fluid; a evacuation portion coupled to the collection portion and configured to receive the collected irrigation fluid and direct the collected irrigation fluid toward an evacuation port included in the evacuation portion; and a straining assembly configured to strain solids/semisolids from the collected irrigation fluid and prevent the solids/semisolids from entering the evacuation port, thus defining strained irrigation fluid; wherein the collection portion is coupled to the drape portion along multiple edges of the drape portion.

One or more of the following features may be included. The limb dam portion may include: a limb passage for at least partially surrounding the limb on which the medical procedure is being performed. The limb dam passage may include: an elastic cinching assembly for cinching around the limb on which the medical procedure is being performed. The straining assembly may include: a fenestrated straining layer configured to strain the solids/semisolids from the collected irrigation fluid and prevent the solids/semisolids from entering the evacuation port, thus defining the strained irrigation fluid. The solids/semisolids in the captured irrigation fluid may include biological solids/semisolids. The evacuation port may be configured to direct the strained irrigation fluid into an external container assembly. The evacuation port may be configured to be releasably coupleable to an external fluid collection device.

In another implementation, an irrigation drape assembly includes: a collection portion configured to be coupleable to a drape portion and collect irrigation fluid produced during a medical procedure on a body portion, thus defining collected irrigation fluid; a evacuation portion coupled to the collection portion and configured to receive the collected irrigation fluid and direct the collected irrigation fluid toward an evacuation port included in the evacuation portion; and a straining assembly configured to strain solids/semisolids from the collected irrigation fluid and prevent the solids/semisolids from entering the evacuation port, thus defining strained irrigation fluid.

One or more of the following features may be included. The collection portion may be coupled to the drape portion along a single edge of the drape portion. The collection portion may be coupled to the drape portion along multiple edges of the drape portion. The straining assembly may include: a fenestrated straining layer configured to strain the solids/semisolids from the captured irrigation fluid and prevent the solids/semisolids from entering the evacuation port, thus defining the strained irrigation fluid. The solids/semisolids in the captured irrigation fluid may include biological solids/semisolids. The evacuation port may be configured to direct the strained irrigation fluid into an external container assembly. The evacuation port may be configured to be releasably coupleable to an external fluid collection device. The collection portion may include an adhesive strip for coupling the collection portion to the drape portion.

The details of one or more implementations are set forth in the accompanying drawings and the description below. Other features and advantages will become apparent from the description, the drawings, and the claims.

### BRIEF DESCRIPTION OF THE DRAWINGS

FIGS. 1-6 are diagrammatic views of an irrigation drape assembly according to an embodiment of the present disclosure.

Like reference symbols in the various drawings indicate like elements.

### DETAILED DESCRIPTION OF THE PREFERRED EMBODIMENTS

### System Overview

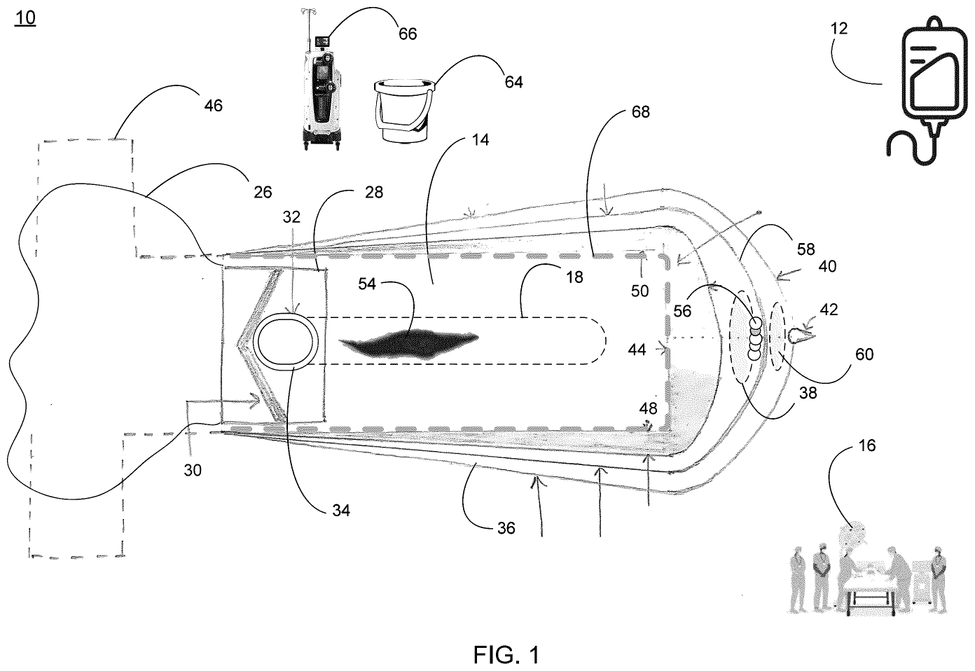

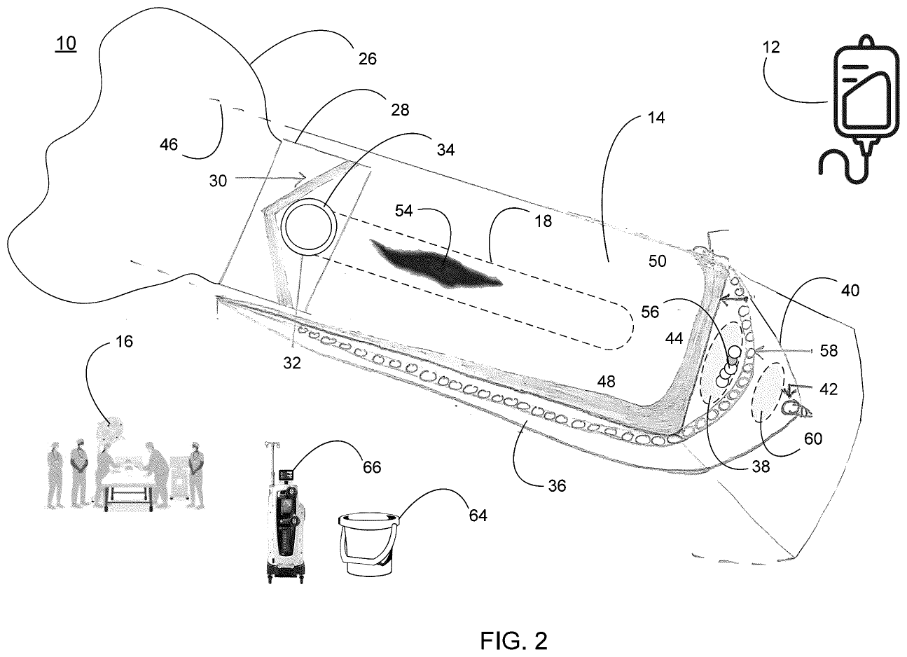

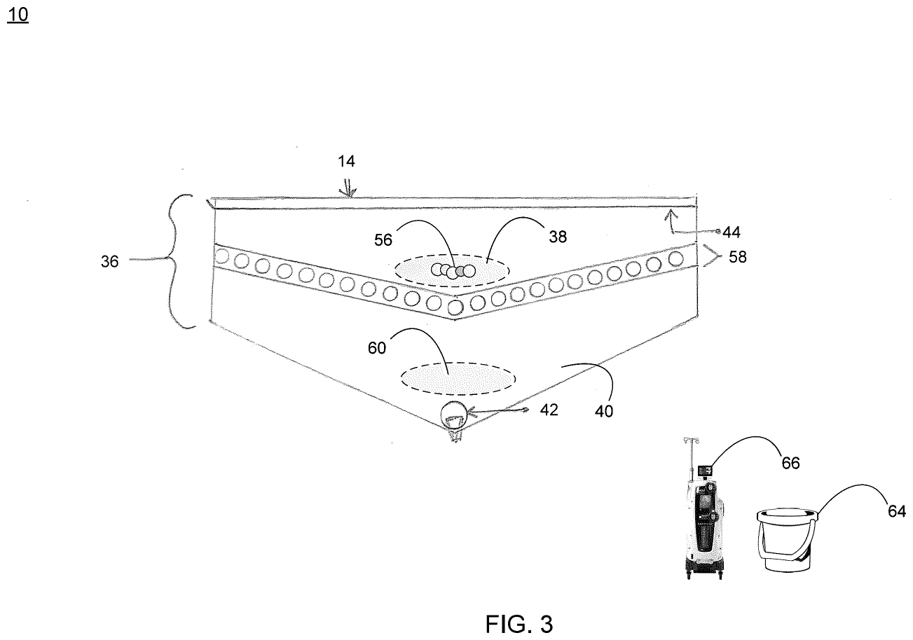

Referring to FIGS. 1-3, there is shown an irrigation drape assembly (e.g., irrigation drape assembly 10). As will be discussed below in greater detail, irrigation drape assembly (e.g., irrigation drape assembly 10) may allow for the irrigation of wounds/injuries using an irrigation fluid (e.g., irrigation fluid 12) while minimizing the risk of spillage (e.g., of irrigation fluid 12) onto the floor of the treatment theater. Examples of the irrigation fluid (e.g., irrigation fluid 12) may include but are not limited to sterile saline solution or sterile water,

Irrigating open wounds is a fundamental step in wound care, and it serves several critical purposes that collectively enhance healing and prevent complications. When a wound is left untreated or inadequately cleaned, dirt, bacteria, dead tissue, and other contaminants can quickly accumulate on the wound surface. Irrigation helps to flush out these harmful elements, ensuring a cleaner wound bed and lowering the risk of infection. By removing this debris, irrigation also allows healthcare providers to work with a clearer view of the wound's condition, enabling them to assess its depth, potential tissue damage, and any specific treatment requirements more accurately.

A major benefit of wound irrigation is its role in preventing infection. Open wounds are highly susceptible to bacterial invasion, which can lead to infections that compromise the healing process and may even result in serious complications, such as cellulitis or sepsis, if left unchecked. The act of cleansing the wound with sterile solutions, typically saline, works to remove or significantly reduce the number of bacteria present. This reduction in bacteria helps create a safer environment for the wound to heal without the interference of pathogenic organisms.

Additionally, irrigation helps to maintain an optimal moisture balance in the wound bed. When wounds dry out, the healing process can stall, and tissue is more likely to become desiccated and damaged, which may lead to slower healing and increased scarring. Proper moisture helps keep the cells that contribute to repair, such as fibroblasts and keratinocytes, active and promotes a smooth, even healing process.

Ultimately, wound irrigation serves as a preparatory step that optimizes the wound environment for healing. By cleaning the wound, preventing infection, and supporting cellular repair, irrigation improves the body's ability to close the wound efficiently and reduces the likelihood of complications or long-term tissue damage.

Generally speaking, the quantity of irrigation fluid used to irrigate a wound depends on various factors, including the wound's size, location, and the level of contamination. For small wounds, such as abrasions or minor lacerations, about 50 to 100 mL of sterile saline or water is often sufficient to remove debris and bacteria. In the case of moderate wounds, such as larger cuts or post-surgical wounds, healthcare providers typically use between 250 and 500 mL to ensure thorough cleansing. For large or heavily contaminated wounds, 500 to 1000 mL or more may be necessary to achieve adequate irrigation. General recommendations suggest using at least 100 to 200 mL of fluid per centimeter of wound length for more serious wounds. This approach helps ensure that contaminants are fully flushed out, reducing the risk of infection and promoting a more optimal healing environment. Saline is generally preferred as it is isotonic and gentle on tissues, minimizing irritation while effectively removing bacteria and debris.

The irrigation drape assembly (e.g., irrigation drape assembly 10) may include: a drape portion (e.g., drape portion 14) configured to be positioned beneath a body portion on which a medical procedure (e.g., medical procedure 16) is being performed.

Examples of the medical procedure (e.g., medical procedure 16) may include but are not limited to the treatment of tissue injuries, muscle injuries, compound fractures, burns, joint injuries, etc. As discussed above, examples of such irrigation fluids may include but are not limited to sterile saline solution or sterile water.

In one implementation, the drape portion (e.g., drape portion 14) may be configured to be positioned beneath a limb (e.g., limb 18) on which the medical procedure (e.g., medical procedure 16) is being performed. For example, the limb (e.g., limb 18) may be the leg of a patient that was involved in a motorcycle crash and experienced a compound fracture in which portions of the femur are protruding through the skin. Accordingly, and in such a situation, irrigation and sterilization with the irrigation fluid (e.g., irrigation fluid 12) is of paramount importance when treating such an open wound/injury.

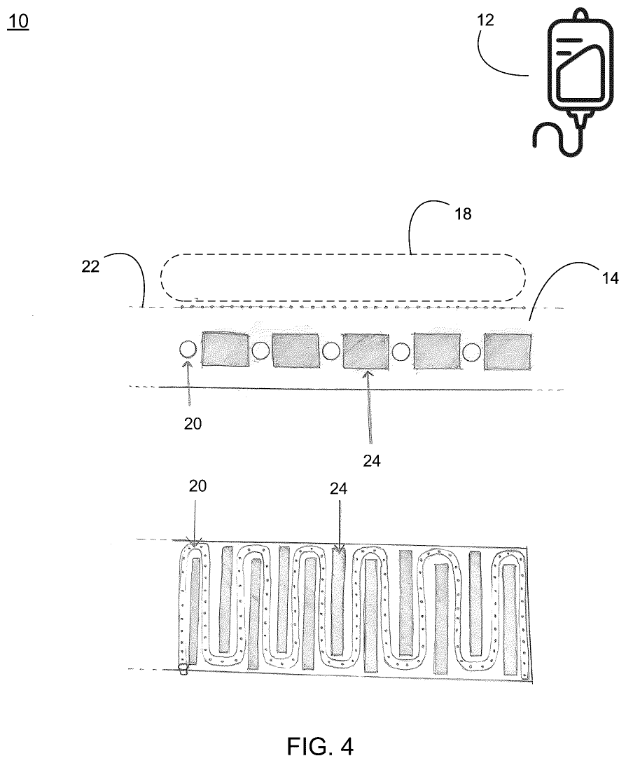

Referring to FIG. 4 and in certain implementations, the drape portion (e.g., drape portion 14) may be porous in nature and, when positioned beneath a limb (e.g., limb 18) on which the medical procedure (e.g., medical procedure 16) is being performed, such porosity may assist in wicking the irrigation fluid (e.g., irrigation fluid 12) away from the limb (e.g., limb 18). For example, the drape portion (e.g., drape portion 14) may include drainage tubing (e.g., drainage tubing 20) positioned below a porous layer (e.g., porous layer 22) of the drape portion (e.g., drape portion 14), with various support structures (e.g., support structures 24) positioned therebetween, so that the irrigation fluid (e.g., irrigation fluid 12) that passes through this porous layer (e.g., porous layer 22) may be collected within the drainage tubing (e.g., drainage tubing 20) and evacuated to e.g., an external container or a fluid collection system.

In certain implementations, the drape portion (e.g., drape portion 14) may include an upper covering portion (e.g., upper covering portion 26) configured to cover all or a portion of the patient that is not subject to the medical procedure (e.g., medical procedure 16), thus preserving the sterile nature of the operating environment.

When configured to be used with a limb (e.g., limb 18), the drape portion (e.g., drape portion 14) may include: a limb dam portion (e.g., limb dam portion 28). Such a limb dam portion (e.g., limb dam portion 28) may be configured to prevent longitudinal spillage of the above-referenced irrigation fluid (e.g., irrigation fluid 12) along the length of the limb (e.g., limb 18).

The limb dam portion (e.g., limb dam portion 28) may include one or more fluid direction ridges (e.g., fluid direction ridges 30) to prevent the irrigation fluid (e.g., irrigation fluid 12) from moving e.g., toward the torso of the patient upon which the medical procedure (e.g., medical procedure 16) is being performed, thus reducing the risk of spilling of irrigation fluid 12 onto the floor of the treatment theater.

This limb dam portion (e.g., limb dam portion 28) may include one or more limb passages. For example, limb dam portion (e.g., limb dam portion 28) may include: a limb passage (e.g., limb passage 32 for at least partially surrounding the limb (e.g., limb 18) on which the medical procedure (e.g., medical procedure 16) is being performed.

For example, this limb passage (e.g., limb passage 32) may fully surround the limb (e.g., limb 18) on which the medical procedure (e.g., medical procedure 16 is being performed. Such a limb passage (e.g., limb passage 32) may be a hole within the limb dam portion (e.g., limb dam portion 28) through which the limb (e.g., limb 18) passes.

In certain implementations when the limb passage (e.g., limb passage 32) fully surrounds the limb (e.g., limb 18), this limb passage (e.g., limb passage 32) may include: an elastic cinching assembly (e.g., elastic cinching assembly 34) for cinching around the limb (e.g., limb 18) on which the medical procedure (e.g., medical procedure 16) is being performed. For example, the periphery of this limb passage (e.g., limb passage 32) may be constructed of an elastic material that forms the elastic cinching assembly (e.g., elastic cinching assembly 34) for sealing around the surface of the limb (e.g., limb 18). Examples of this elastic material may include but are not limited to elastin, rubber, nylon, lycra, silicon and polyester.

The irrigation drape assembly (e.g., irrigation drape assembly 10) may also include: a collection portion (e.g., collection portion 36) configured to collect the irrigation fluid (e.g., irrigation fluid 12) utilized during the medical procedure (e.g., medical procedure 16) on the body portion, thus defining collected irrigation fluid (e.g., collected irrigation fluid 38). The collection portion (e.g., collection portion 36) may be configured to ensure that the irrigation fluid (e.g., irrigation fluid 12) utilized during the medical procedure (e.g., medical procedure 16) flows into the collection portion (e.g., collection portion 36). For example, one or more rigid edges or spacer assemblies may be included within collection portion 36 to form a port into which irrigation fluid 12 will flow.

The irrigation drape assembly (e.g., irrigation drape assembly 10) may also include: an evacuation portion (e.g., evacuation portion 40) coupled to the collection portion (e.g., collection portion 36) and configured to receive the collected irrigation fluid (e.g., collected irrigation fluid 38). The evacuation portion (e.g., evacuation portion 40) may direct the collected irrigation fluid (e.g., collected irrigation fluid 38) toward an evacuation port (e.g., evacuation port 42) included in the evacuation portion (e.g., evacuation portion 40).

In certain embodiments, the collection portion (e.g., collection portion 36) may be coupled to the drape portion (e.g., drape portion 14) along a single edge (e.g., edge 44) of the drape portion (e.g., drape portion 14), wherein the evacuation port (e.g., evacuation port 42) may be positioned opposite the single edge (e.g., edge 44).

For example, the drape portion (e.g., drape portion 14) may be positioned under the limb (e.g., limb 18) on which the medical procedure (e.g., medical procedure 16) is being performed. The drape portion (e.g., drape portion 14) may be positioned generally horizontal and placed on top of the examination table (e.g., examination table 46) on which the patient and the limb (e.g., limb 18) is positioned.

The collection portion (e.g., collection portion 36) may be coupled to the drape portion (e.g., drape portion 14) along a single edge (e.g., edge 44) of the drape portion (e.g., drape portion 14). The collection portion (e.g., collection portion 36) may hang over the side of the examination table (e.g., examination table 46), thus enabling the collected irrigation fluid (e.g., collected irrigation fluid 38) to cascade over the single edge (e.g., edge 44) and flow “downstream” toward the evacuation port (e.g., evacuation port 42) included in the evacuation portion (e.g., evacuation portion 40).

In other embodiments, the collection portion (e.g., collection portion 36) may be coupled to the drape portion (e.g., drape portion 14) along multiple edges (e.g., multiple edges 44, 48, 50) of the drape portion (e.g., drape portion 14).

For example, the drape portion (e.g., drape portion 14) may be positioned under the limb (e.g., limb 18) on which the medical procedure (e.g., medical procedure 16) is being performed. The drape portion (e.g., drape portion 14) may be positioned generally horizontal and placed on top of the examination table (e.g., examination table 46) on which the patient and the limb (e.g., limb 18) is positioned. The collection portion (e.g., collection portion 36) may be coupled to the drape portion (e.g., drape portion 14) along multiple edges (e.g., multiple edges 44, 48, 50) of the drape portion (e.g., drape portion 14). Again, the collection portion (e.g., collection portion 36) may hang over the sides of the examination table (e.g., examination table 46), thus enabling the collected irrigation fluid (e.g., collected irrigation fluid 38) to cascade over multiple edges (e.g., multiple edges 44, 48, 50) and flow “downstream” toward the evacuation port (e.g., evacuation port 42) included in the evacuation portion (e.g., evacuation portion 40).

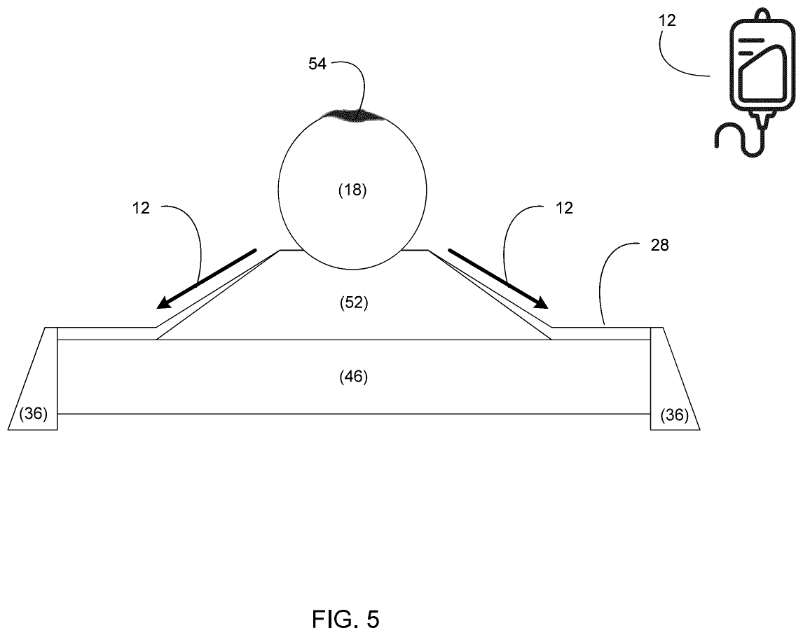

Referring also to FIG. 5 and in some embodiments, the drape portion (e.g., drape portion 14) may be placed upon an elevation platform (e.g., elevation platform 52) to raise the limb (e.g., limb 18) above the examination table (e.g., examination table 46) on which the patient and the limb (e.g., limb 18) is positioned. This elevated position may assist in directing the irrigation fluid (e.g., irrigation fluid 12) away from the limb (e.g., limb 18) and toward the collection portion (e.g., collection portion 36).

As one of the reasons for irrigating a wound/injury (e.g., wound 54) is to remove debris (e.g., dirt, glass, blood, tissue, muscle, bone), it is foreseeable that the captured irrigation fluid (e.g., captured irrigation fluid 38) may include biological solids/semisolids (e.g., solids/semisolids 56). Therefore, the solids/semisolids (e.g., solids/semisolids 56) in the collected irrigation fluid (e.g., collected irrigation fluid 38) may include biological solids/semisolids (e.g., solids/semisolids 56).

Accordingly, the irrigation drape assembly (e.g., irrigation drape assembly 10) may include: a straining assembly (e.g., straining assembly 58) configured to strain solids/semisolids (e.g., solids/semisolids 56) from the collected irrigation fluid (e.g., collected irrigation fluid 38) and prevent the solids/semisolids (e.g., solids/semisolids 56) from entering the evacuation port (e.g., evacuation port 42), thus defining strained irrigation fluid (e.g., strained irrigation fluid 60).

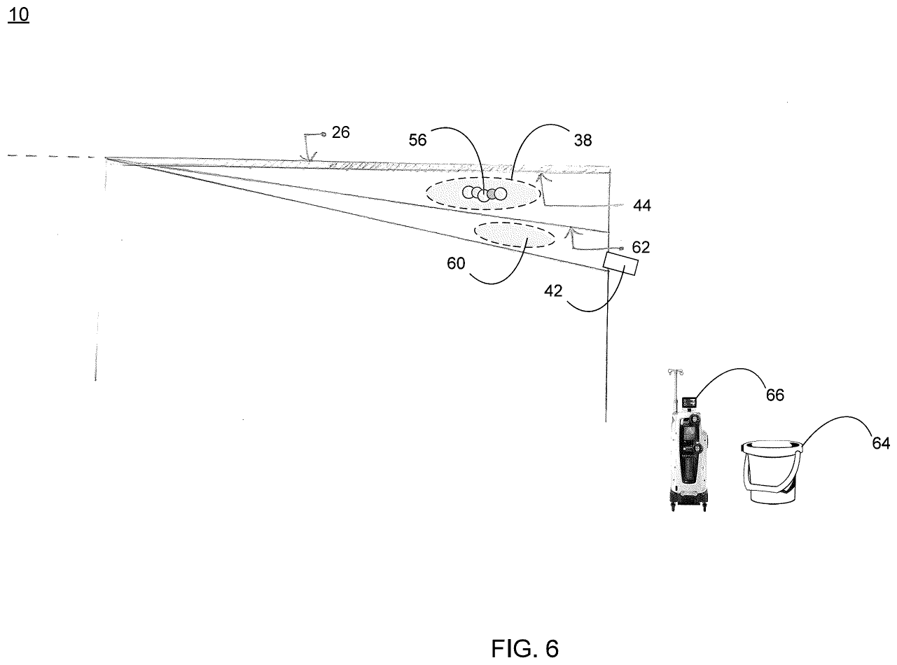

Referring also to FIG. 6 and in one implementation, the straining assembly (e.g., straining assembly 58) may include: a fenestrated straining layer (e.g., fenestrated straining layer 62) configured to strain the solids/semisolids (e.g., solids/semisolids 56) from the collected irrigation fluid (e.g., collected irrigation fluid 38) and prevent the solids/semisolids (e.g., solids/semisolids 56) from entering the evacuation port (e.g., evacuation port 42).

The evacuation port (e.g., evacuation port 42) may be configured to direct the strained irrigation fluid (e.g., strained irrigation fluid 60) into an external container assembly (e.g., external container assembly 64). An example of such an external container assembly (e.g., external container assembly 64) may include but is not limited to a bucket/container assembly.

Additionally/alternatively, the evacuation port (e.g., evacuation port 42) may be configured to be releasably coupleable to an external fluid collection device (e.g., external fluid collection device 66). Examples of such an external fluid collection device (e.g., external fluid collection device 66) may include but is not limited to the Stryker Neptune 3 Waste Management System.

In an alternative embodiment, the collection portion (e.g., collection portion 36), the evacuation portion (e.g., evacuation portion 40) and the straining assembly (e.g., straining assembly 58) of the irrigation drape assembly (e.g., irrigation drape assembly 10) may be available without the drape portion (e.g., drape portion 14). Accordingly and in such an embodiment, the user of the irrigation drape assembly (e.g., irrigation drape assembly 10) may use it with whatever drape portion they wish to use or are accustomed to using.

Accordingly and in such an alternative embodiment, the irrigation drape assembly (e.g., irrigation drape assembly 10) may include: a collection portion (e.g., collection portion 36) configured to be coupleable to a drape portion (e.g., drape portion 14). The collection portion (e.g., collection portion 36) may be configured to receive irrigation fluid (e.g., irrigation fluid 12) collected by the drape portion (e.g., drape portion 14), wherein this irrigation fluid (e.g., irrigation fluid 12) may be directed toward an evacuation port (e.g., evacuation port 42) included in the evacuation portion (e.g., evacuation portion 40).

The collection portion (e.g., collection portion 36) may include an adhesive strip (e.g., adhesive strip 68) for coupling the collection portion (e.g., collection portion 36) to the drape portion (e.g., drape portion 14). Accordingly and in such a configuration, being this collection portion (e.g., collection portion 36) is coupleable to the drape portion (e.g., drape portion 14) via e.g., adhesive strip 68, this collection portion (e.g., collection portion 36) and evacuation portion (e.g., evacuation portion 40) may be utilized with a variety of readily-available drape portions (e.g., drape portion 14).

As discussed above, the collection portion (e.g., collection portion 36) may be coupled to the drape portion (e.g., drape portion 14) along a single edge (e.g., edge 44) of the drape portion (e.g., drape portion 14).

As also discussed above, the collection portion (e.g., collection portion 36) may be coupled to the drape portion (e.g., drape portion 14) along multiple edges (e.g., multiple edges 44, 48, 50) of the drape portion (e.g., drape portion 14).

As discussed above, the irrigation drape assembly (e.g., irrigation drape assembly 10) may include: a straining assembly (e.g., straining assembly 58) configured to strain solids/semisolids (e.g., solids/semisolids 56) from the collected irrigation fluid (e.g., collected irrigation fluid 38) and prevent the solids/semisolids (e.g., solids/semisolids 56) from entering the evacuation port (e.g., evacuation port 42) within the evacuation portion (e.g., evacuation portion 40), thus defining strained irrigation fluid (e.g., strained irrigation fluid 60). The solids/semisolids (e.g., solids/semisolids 56) in the collected irrigation fluid (e.g., collected irrigation fluid 38) may include biological solids/semisolids (e.g., solids/semisolids 56).

As also discussed above, the straining assembly (e.g., straining assembly 58) may include: a fenestrated straining layer (e.g., fenestrated straining layer 62) configured to strain the solids/semisolids (e.g., solids/semisolids 56) from the collected irrigation fluid (e.g., collected irrigation fluid 38) and prevent the solids/semisolids (e.g., solids/semisolids 56) from entering the evacuation port (e.g., evacuation port 42).

As also discussed above, the evacuation port (e.g., evacuation port 42) may be configured to direct the strained irrigation fluid (e.g., strained irrigation fluid 60) into an external container assembly (e.g., external container assembly 64).

As also discussed above, the evacuation port (e.g., evacuation port 42) may be configured to be releasably coupleable to an external fluid collection device (e.g., external fluid collection device 66).

### General

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. An irrigation drape assembly comprising:
a drape portion configured to be positioned beneath a body portion on which a medical procedure is being performed;
a collection portion configured to collect irrigation fluid produced during the medical procedure on the body portion, thus defining collected irrigation fluid;
a evacuation portion coupled to the collection portion and configured to receive the collected irrigation fluid and direct the collected irrigation fluid toward an evacuation port included in the evacuation portion; and
a straining assembly configured to strain solids/semisolids from the collected irrigation fluid and prevent the solids/semisolids from entering the evacuation port, thus defining strained irrigation fluid.

2. The irrigation drape assembly of claim 1 wherein the body portion is a limb on which the medical procedure is being performed.

3. The irrigation drape assembly of claim 2 wherein the drape portion includes:
a limb dam portion.

4. The irrigation drape assembly of claim 3 wherein the limb dam portion includes:
a limb passage for at least partially surrounding the limb on which the medical procedure is being performed.

5. The irrigation drape assembly of claim 4 wherein the limb dam passage includes:
an elastic cinching assembly for cinching around the limb on which the medical procedure is being performed.

6. The irrigation drape assembly of claim 1 wherein the collection portion is coupled to the drape portion along a single edge of the drape portion.

7. The irrigation drape assembly of claim 1 wherein the collection portion is coupled to the drape portion along multiple edges of the drape portion.

8. The irrigation drape assembly of claim 1 wherein the straining assembly includes:
a fenestrated straining layer configured to strain the solids/semisolids from the collected irrigation fluid and prevent the solids/semisolids from entering the evacuation port, thus defining the strained irrigation fluid.

9. The irrigation drape assembly of claim 1 wherein the solids/semisolids in the captured irrigation fluid includes biological solids/semisolids.

10. The irrigation drape assembly of claim 1 wherein the evacuation port is configured to direct the strained irrigation fluid into an external container assembly.

11. The irrigation drape assembly of claim 1 wherein the evacuation port is configured to be releasably coupleable to an external fluid collection device.

12. An irrigation drape assembly comprising:
a drape portion configured to be positioned beneath a limb on which a medical procedure is being performed, the drape portion including a limb dam portion;
a collection portion configured to collect irrigation fluid produced during the medical procedure on the limb, thus defining collected irrigation fluid;
a evacuation portion coupled to the collection portion and configured to receive the collected irrigation fluid and direct the collected irrigation fluid toward an evacuation port included in the evacuation portion; and
a straining assembly configured to strain solids/semisolids from the collected irrigation fluid and prevent the solids/semisolids from entering the evacuation port, thus defining strained irrigation fluid;
wherein the collection portion is coupled to the drape portion along multiple edges of the drape portion.

13. The irrigation drape assembly of claim 12 wherein the limb dam portion includes:
a limb passage for at least partially surrounding the limb on which the medical procedure is being performed.

14. The irrigation drape assembly of claim 13 wherein the limb dam passage includes:
an elastic cinching assembly for cinching around the limb on which the medical procedure is being performed.

15. The irrigation drape assembly of claim 12 wherein the straining assembly includes:
a fenestrated straining layer configured to strain the solids/semisolids from the collected irrigation fluid and prevent the solids/semisolids from entering the evacuation port, thus defining the strained irrigation fluid.

16. The irrigation drape assembly of claim 12 wherein the solids/semisolids in the captured irrigation fluid includes biological solids/semisolids.

17. The irrigation drape assembly of claim 12 wherein the evacuation port is configured to direct the strained irrigation fluid into an external container assembly.

18. The irrigation drape assembly of claim 12 wherein the evacuation port is configured to be releasably coupleable to an external fluid collection device.

19. An irrigation drape assembly comprising:
a collection portion configured to be coupleable to a drape portion and collect irrigation fluid produced during a medical procedure on a body portion, thus defining collected irrigation fluid;
a evacuation portion coupled to the collection portion and configured to receive the collected irrigation fluid and direct the collected irrigation fluid toward an evacuation port included in the evacuation portion; and
a straining assembly configured to strain solids/semisolids from the collected irrigation fluid and prevent the solids/semisolids from entering the evacuation port, thus defining strained irrigation fluid.

20. The irrigation drape assembly of claim 19 wherein the collection portion is coupled to the drape portion along a single edge of the drape portion.

21. The irrigation drape assembly of claim 19 wherein the collection portion is coupled to the drape portion along multiple edges of the drape portion.

22. The irrigation drape assembly of claim 19 wherein the straining assembly includes:
a fenestrated straining layer configured to strain the solids/semisolids from the captured irrigation fluid and prevent the solids/semisolids from entering the evacuation port, thus defining the strained irrigation fluid.

23. The irrigation drape assembly of claim 19 wherein the solids/semisolids in the captured irrigation fluid includes biological solids/semisolids.

24. The irrigation drape assembly of claim 19 wherein the evacuation port is configured to direct the strained irrigation fluid into an external container assembly.

25. The irrigation drape assembly of claim 19 wherein the evacuation port is configured to be releasably coupleable to an external fluid collection device.

26. The irrigation drape assembly of claim 19 wherein the collection portion includes an adhesive strip for coupling the collection portion to the drape portion.
